(19) United States
(12) Patent Application Publication
(10) Pub. No.: US 2021/0401358 A1
(43) Pub. Date: Dec. 30, 2021

(54) HEALTH CARE PROVIDER AUTHORIZATION OF DATA ACQUISITION BY SENSOR ENABLED WOUND DRESSINGS AND DEVICES

(71) Applicant: Smith & Nephew PLC, Watford, Hertfordshire (GB)

(72) Inventors: Felix Clarence QUINTANAR, Hull (GB); Johannes Dagevos VAN RIJ, Cottingham (GB)

(21) Appl. No.: 17/293,442

(22) PCT Filed: Nov. 13, 2019
(86) PCT No.: PCT/EP2019/081248

§ 371 Date: May 12, 2021

(30) Foreign Application Priority Data
Nov. 14, 2018 (GB) 1818552.0

(51) Int. Cl. A61B 5/00; A61B 5/01; A61B 5/145; A61B 5/1455; A61B 5/0531; A61F 13/00

(52) U.S. Cl. CPC A61B 5/445; A61B 5/01; A61B 5/6802; A61B 5/14539; A61B 5/14551; A61B 2562/046; A61B 5/0004; A61F 13/00068; A61B 2562/0271; A61B 2562/0247; A61B 5/0531

(57) ABSTRACT

In some embodiments, a wound monitoring and/or treatment system includes a dressing and/or housing configured to be placed in or over wound and/or skin of a patient, sensor configured to measure patient data, and controller configured to receive patient data measured by the sensor, selectively store at least some of the patient data in a memory, and communicate, via a transceiver, at least some of the data stored in the memory to an external computing device. The controller can be configured to, in response to determining that the dressing and/or housing is placed in or over the wound and/or skin, communicate, via the transceiver, to the external computing device a confirmation. The controller can be configured to, in response to receiving, via the transceiver, from the external computing device an authorization to collect patient data, store at least some of the patient data measured by the sensor in the memory.

### TECHNICAL FIELD

Embodiments of the present disclosure relate to methods and apparatuses for monitoring and/or treating a wound with, for example with reduced pressure therapy or topical negative pressure (TNP) therapy. In particular, but without limitation, embodiments disclosed herein relate to the acquisition of data by sensor enabled wound dressings.

### DESCRIPTION OF THE RELATED ART

Many different types of wound dressings are known for aiding in the healing process of a human or animal. These different types of wound dressings include many different types of materials and layers, for example, gauze, pads, foam pads or multi-layer wound dressings. Topical negative pressure (TNP) therapy, sometimes referred to as vacuum assisted closure, negative pressure wound therapy, or reduced pressure wound therapy, is widely recognized as a beneficial mechanism for improving the healing rate of a wound. Such therapy is applicable to a broad range of wounds such as incisional wounds, open wounds, and abdominal wounds or the like.

TNP therapy assists in the closure and healing of wounds by reducing tissue edema, encouraging blood flow, stimulating the formation of granulation tissue, removing excess exudates and may reduce bacterial load. Thus, reducing infection to the wound. Furthermore, TNP therapy permits less outside disturbance of the wound and promotes more rapid healing.

For wound monitoring and/or therapy to be effective, it can be advantageous to obtain various patient data. There exists a need to safely and effectively obtain patient data.

### SUMMARY

A wound monitoring and/or treatment system can include a dressing, housing, or dressing and housing that can be configured to be placed in or over a wound, skin, or wound and skin of a patient; a sensor positioned on or in the dressing, housing, or dressing and housing and can be configured to measure patient data; a transceiver positioned on or in the dressing, housing, or dressing and housing; and a controller positioned on or in the dressing, housing, or dressing and housing. The sensor can comprise at least one of a pressure sensor, conductivity sensor, blood oxygen saturation sensor, optical sensor, pH sensor, temperature sensor, or motion sensor. The controller can be configured to receive patient data measured by the sensor, selectively store at least some of the patient data in a memory, and communicate, via the transceiver, at least some of the data stored in the memory to an external computing device. The controller can be configured to determine based on data measured by the sensor if the dressing, housing, or dressing and housing is placed in or over the wound, skin, or wound and skin of the patient; in response to determining that the dressing, housing, or dressing and housing is placed in or over the wound, skin, or wound and skin of the patient, communicate, via the transceiver, to the external computing device a confirmation that the dressing, housing, or dressing and housing is placed in or over the wound, skin, or wound and skin of the patient and causing the external computing device to receive an authorization from a healthcare provider (HCP) to collect the patient data; and in response to receiving, via the transceiver, from the external computing device the authorization to collect patient data, store at least some of the patient data measured by the sensor in the memory.

A wound monitoring and/or treatment system can include a dressing, housing, or dressing and housing configured to be placed in or over a wound, skin, or wound and skin of a patient. The system can include a sensor positioned on or in the dressing, housing, or dressing and housing and configured to measure patient data. The sensor can include at least one of a pressure sensor, conductivity sensor, blood oxygen saturation sensor, optical sensor, pH sensor, temperature sensor, or motion sensor The system can include a transceiver. The system can include a controller configured to receive patient data measured by the sensor, selectively store at least some of the patient data in a memory, and communicate, via the transceiver, at least some of the data stored in the memory to an external computing device. The controller can be configured to determine based on data measured by the sensor if the dressing, housing, or dressing and housing is placed in or over the wound, skin, or wound and skin of the patient. The controller can be configured to in response to determining that the dressing, housing, or dressing and housing is placed in or over the wound, skin, or wound and skin of the patient, communicate, via the transceiver, to the external computing device a confirmation that the dressing, housing, or dressing and housing is placed in or over the wound, skin, or wound and skin of the patient and causing the external computing device to receive an authorization from a healthcare provider (HCP) to collect the patient data. The controller can be configured to, in response to receiving, via the transceiver, from the external computing device the authorization to collect patient data, store at least some of the patient data measured by the sensor in the memory.

The system any of the preceding paragraphs and/or any other system described herein can include one or more of the following features. The controller can be configured to, in response to not receiving the authorization to collect patient data, prevent storing at least some of the patient data in the memory. The sensor can comprise a temperature sensor and the controller can be configured to determine that the dressing, housing, or dressing and housing is placed in or over the wound, skin, or wound and skin of the patient in response to determining that patient temperature measured by the temperature sensor is within a temperature range. The system can comprise an ambient temperature sensor positioned on or in the dressing, housing, or dressing and housing and can be configured to measure the ambient temperature, wherein the controller can be configured to determine that the dressing, housing, or dressing and housing is placed in or over the wound, skin, or wound and skin of the patient in response to determining that a difference between temperature measured by the pressure sensor and the ambient pressure satisfies a temperature difference threshold. The sensor can comprise a pressure sensor and the controller can be configured to determine that the dressing, housing, or dressing and housing is placed in or over the wound, skin, or wound and skin of the patient in response to determining that pressure measured by the pressure sensor satisfies a pressure threshold. The system can comprise an ambient pressure sensor positioned on or in the dressing, housing, or dressing and housing and can be configured to measure the ambient pressure, wherein the controller can be configured to determine that the dressing, housing, or dressing and housing is placed in or over the wound, skin, or wound and skin of the patient in response to determining that a difference between pressure measured by the sensor and the ambient temperature satisfies a pressure difference threshold. The sensor can comprise a conductivity sensor and the controller can be configured to determine that the dressing, housing, or dressing and housing is placed in or over the wound, skin, or wound and skin of the patient in response to determining that conductivity measured by the conductivity sensor satisfies a conductivity threshold. The sensor can comprise an optical sensor and the controller can be configured to determine that the dressing, housing, or dressing and housing is placed in or over the wound, skin, or wound and skin of the patient in response to determining that image data measured by the optical sensor is associated with image data of a wound, skin, or wound and skin. The sensor can comprise a blood oxygen saturation sensor and the controller can be configured to determine that the dressing, housing, or dressing and housing is placed in or over the wound, skin, or wound and skin of the patient in response to determining that blood oxygen saturation measured by the blood oxygen saturation sensor is within blood oxygen saturation range. The sensor can comprise an pH sensor and the controller can be configured to determine that the dressing, housing, or dressing and housing is placed in or over the wound, skin, or wound and skin of the patient in response to determining that pH level measured by the pH sensor satisfies a pH threshold. The sensor can comprise a motion sensor and the controller can be configured to determine that the dressing, housing, or dressing and housing is placed in or over the wound, skin, or wound and skin of the patient in response to determining that motion data measured by the motion sensor satisfies a motion threshold. The controller can be configured to receive, via the transceiver, from the external computing device an indication by the HCP to stop patient data. A method of operating the system of any of preceding paragraphs or any of following paragraphs is provided.

A method of authorizing collection of patient data can include, by a controller of a dressing, housing, or dressing and housing that can be configured to be placed in or over a wound, skin, or wound and skin of a patient, determining based on data measured by a sensor positioned on or in the dressing, housing, or dressing and housing, the sensor configured to measure patient data, if the dressing, housing, or dressing and housing is placed in or over the wound, skin, or wound and skin of the patient. The method can include, in response to determining that the dressing, housing, or dressing and housing is placed in or over the wound, skin, or wound and skin of the patient, communicating, via a transceiver positioned on or in the dressing, housing, or dressing and housing, to an external computing device a confirmation that the dressing, housing, or dressing and housing is placed in or over the wound, skin, or wound and skin of the patient and causing the external computing device to receive an authorization from a healthcare provider (HCP) to collect the patient data. The method can include, in response to receiving, via the transceiver, from the external computing device the authorization to collect patient data, store at least some of the patient data measured by the sensor in the memory.

A method of authorizing collection of patient data can include, by a controller configured to be in communication with a dressing, housing, or dressing and housing configured to be placed in or over a wound, skin, or wound and skin of a patient, wherein a sensor is positioned on or in the dressing, housing, or dressing and housing, and wherein the sensor is configured to measure patient data, determining based on data measured by the sensor if the dressing, housing, or dressing and housing is placed in or over the wound, skin, or wound and skin of the patient. The method can include, by the controller, in response to determining that the dressing, housing, or dressing and housing is placed in or over the wound, skin, or wound and skin of the patient, communicating, via a transceiver in communication with the controller, to an external computing device a confirmation that the dressing, housing, or dressing and housing is placed in or over the wound, skin, or wound and skin of the patient and causing the external computing device to receive an authorization from a healthcare provider (HCP) to collect the patient data. The method can include, by the controller, in response to receiving, via the transceiver, from the external computing device the authorization to collect patient data, storing at least some of the patient data measured by the sensor in the memory.

The method of any of the preceding paragraphs and/or any other methods described herein can include one or more of the following steps and/or features. The method can comprise in response to not receiving the authorization to collect patient data, preventing storing at least some of the patient data in the memory. The sensor can comprise a temperature sensor and the method can comprise determining that the dressing, housing, or dressing and housing is placed in or over the wound, skin, or wound and skin of the patient in response to determining that patient temperature measured by the temperature sensor is within a temperature range. An ambient temperature sensor that can be positioned on or in the dressing, housing, or dressing and housing and can be configured to measure the ambient temperature. The method can comprise determining that the dressing, housing, or dressing and housing is placed in or over the wound, skin, or wound and skin of the patient in response to determining that a difference between temperature measured by the sensor and the ambient temperature satisfies a temperature difference threshold. The sensor can comprise a pressure sensor and the method can comprise determining that the dressing, housing, or dressing and housing is placed in or over the wound, skin, or wound and skin of the patient in response to determining that pressure measured by the pressure sensor satisfies a pressure threshold. An ambient pressure sensor that can be positioned on or in the dressing, housing, or dressing and housing and can be configured to measure the ambient pressure. The method can comprise determining that the dressing, housing, or dressing and housing is placed in or over the wound, skin, or wound and skin of the patient in response to determining that a difference between pressure measured by the pressure sensor and the ambient pressure satisfies a pressure difference threshold. The sensor can comprise a conductivity sensor and the method can comprise determining that the dressing, housing, or dressing and housing is placed in or over the wound, skin, or wound and skin of the patient in response to determining that conductivity measured by the conductivity sensor satisfies a conductivity threshold. The sensor can comprise an optical sensor and the method can comprise determining that the dressing, housing, or dressing and housing is placed in or over the wound, skin, or wound and skin of the patient in response to determining that image data measured by the optical sensor is associated with image data of a wound, skin, or wound and skin. The sensor can comprise a blood oxygen saturation sensor and the method can comprise determining that the dressing, housing, or dressing and housing is placed in or over the wound, skin, or wound and skin of the patient in response to determining that blood oxygen saturation measured by the blood oxygen saturation sensor is within blood oxygen saturation range. The sensor can comprise a pH sensor and the method can comprise determining that the dressing, housing, or dressing and housing is placed in or over the wound, skin, or wound and skin of the patient in response to determining that pH level measured by the pH sensor satisfies a pH threshold. The sensor can comprise a motion sensor and the method can comprise determining that the dressing, housing, or dressing and housing is placed in or over the wound, skin, or wound and skin of the patient in response to determining that motion data measured by the motion sensor satisfies a motion threshold. The method can comprise receiving, via the transceiver, from the external computing device an indication by the HCP to stop patient data collection; and in response to receiving the indication, prevent storing in memory at least some of the patient data received from the sensor subsequent to the indication. The authorization can include a first time stamp indicating a start of a patient data collection episode. The indication can include a second time stamp indicating an end of the patient data collection episode, and wherein the method can comprise associating patient data stored after receiving the first time stamp and before receiving the second time stamp as being associated with the patient data collection episode.

### BRIEF DESCRIPTION OF THE DRAWINGS

FIG. 1A illustrates a sensor enabled wound dressing.

FIG. 1B illustrates a sensor array illustrating the sensor placement incorporated into a wound dressing.

FIG. 2A illustrates a monitoring device on a body part.

FIG. 2B illustrates a monitoring device.

FIG. 3 illustrates a wound dressing incorporating a source of negative pressure and/or other electronic assembly with sensors within the wound dressing.

FIG. 4 illustrates an exploded view of the housing of the electronics assembly used in FIG. 3.

FIG. 5 illustrates a schematic of a system which can be employed in the embodiments described herein.

FIG. 6 illustrates a flow chart of a process for authorizing patient data collection.

### DETAILED DESCRIPTION

Embodiments disclosed herein relate to systems and methods of monitoring and/or treating a wound. It will be appreciated that throughout this specification reference is made to a wound. It is to be understood that the term wound is to be broadly construed and encompasses open and closed wounds in which skin is torn, cut or punctured or where trauma causes a contusion, or any other superficial or other conditions or imperfections on the skin of a patient or otherwise that benefit from reduced pressure treatment. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

Embodiments of systems and methods disclosed herein can be used with topical negative pressure (“TNP”) or reduced pressure therapy systems. Briefly, negative pressure wound therapy assists in the closure and healing of many forms of “hard to heal” wounds by reducing tissue oedema, encouraging blood flow and granular tissue formation, and/or removing excess exudate and can reduce bacterial load (and thus infection risk). In addition, the therapy allows for less disturbance of a wound leading to more rapid healing. TNP therapy systems can also assist in the healing of surgically closed wounds by removing fluid. In some embodiments, TNP therapy helps to stabilize the tissue in the apposed position of closure. A further beneficial use of TNP therapy can be found in grafts and flaps where removal of excess fluid is important and close proximity of the graft to tissue is required in order to ensure tissue viability.

As is used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, etc.). Accordingly, a negative pressure value of −X mmHg reflects absolute pressure that is X mmHg below 760 mmHg or, in other words, an absolute pressure of (760−X) mmHg In addition, negative pressure that is “less” or “smaller” than X mmHg corresponds to pressure that is closer to atmospheric pressure (for example, −40 mmHg is less than −60 mmHg). Negative pressure that is “more” or “greater” than −X mmHg corresponds to pressure that is further from atmospheric pressure (for example, −80 mmHg is more than −60 mmHg). In some embodiments, local ambient atmospheric pressure is used as a reference point, and such local atmospheric pressure may not necessarily be, for example, 760 mmHg.

Systems and methods disclosed herein can be used with other types of treatment in addition to or instead of reduced pressure therapy, such as irrigation, ultrasound, heat and/or cold, neuro stimulation, or the like. In some cases, disclosed systems and methods can be used for wound monitoring without application of additional therapy. Systems and methods disclosed herein can be used in conjunction with a dressing, including with compression dressing, reduced pressure dressing, or the like.

### Sensor Types

Embodiments described herein can use one or more sensors as described herein. The one or more sensors can be positioned in or on the wound (which, as described herein, can include skin) of a patient to collect patient information or data. Collected patient information can be processed to determine status of the wound in or on which the one or more sensors are positioned and/or to provide treatment. The one or more sensors can include temperature sensors, conductivity sensors, blood oxygen saturation sensors, pulse sensors, optical sensors, pressure sensors, pH sensors, motion sensors, or the like. The one or more sensors can be deployed individually or on a sensor array. Collected and/or processed patient data can assist a clinician in monitoring the status and/or healing of the wound. The one or more sensors can operate individually or in coordination with each other to provide data relating to the wound and/or wound healing characteristics. Collecting data from the wounds that heal well and from those that do not can provide useful insights towards identifying measures to indicate whether a wound is on a healing trajectory.

Temperature sensors can use thermocouples and/or thermistors to measure temperature. Temperature sensors can be used to measure or track the temperature of the underlying wound or the thermal environment within the wound, such as under a dressing. Temperature sensors (or any other sensors disclosed herein) can be calibrated and the data obtained from the sensors can be processed to provide information about the wound environment. An ambient temperature sensor measuring ambient air temperature can be used to assist, for example, with eliminating problems associated with environment temperature shifts. For instance, a first temperature sensor can measure temperature of a wound and a second temperature sensor can measure the ambient temperature.

One or more optical sensors can be used to image a wound. For example, a white light or red-green-blue (RGB) sensor with an illumination source can be used. The sensor and the illumination source can be pressed up against the wound tissue such that light penetrates into the tissue and the sensor measures data related to visual appearance of the wound tissue.

In some cases, ultra bright light emitting diodes (LEDs), an RGB sensor, and polyester optical filters can be used as components of the optical sensors to measure through tissue color differentiation. For example, because surface color can be measured from reflected light, a color can be measured from light which has passed through the tissue first for a given geometry. This can include color sensing from diffuse scattered light, from an LED in contact with the skin. An LED can be used with an RGB sensor nearby to detect the light which has diffused through the tissue. The optical sensors can image with diffuse internal light or surface reflected light.

Additionally or alternatively, the optical sensors can be used to measure autofluorescence. Autoflourescense can be manifested by the tissue absorbing light at one wavelength, and emitting at another. Additionally, dead tissue may not auto-fluoresce and so this could be a very strong indication as to if the tissue is healthy or not. Due to blue light (or UV light) having such a short penetration depth, it may be very useful for example to have a UV light with a red sensitive photodiode nearby (or some other wavelength shifted band) to act as a binary test for healthy tissue, which would auto-fluoresce at a very particular wavelength.

One or more conductivity sensors can be used to determine tissue conductance or impedance. In some cases, there can exist difference between living and dead tissue or to changes in impedance due to a wound being opened up in morbid tissue. Conductivity sensors can include one or more excitation and detection electrodes (such as, Ag/AgCl electrodes). The conductivity sensors can be used to measure the change of impedance of a region of wound growth by measuring the impedance of the surrounding tissue/area. In some cases, the change in conductivity on perimeter electrodes due to a wound size or wound shape change can be measured. Conductivity sensors can be used in the wound bed or on the perimeter of the wound.

Impedance measurement can be based on an AC measurement. An excitation signal can be coupled to the tissue capacitively through a sensor or pad with insulating coating. A second similar electrode can be placed some distance away and connected to ground. By applying an excitation signal, an AC current flows through the tissue between the pads.

Second pair(s) of electrodes can be placed between the excitation electrodes, and can be used to sense voltage. These two electrodes are can each be connected to a high impedance amplifiers, whose outputs can be fed to a differential amplifier. By measuring this output voltage, and dividing by the excitation current, the impedance between the measurement electrodes can be measured.

One or more pH sensors can be used. A pH sensor can include one or more pH changing pads. A spectrometer and a broadband white light source can be used to measure the spectral response of the pH dye. Illumination and imaging can be provided on the surface of a dressing or a housing that is in contact with the wound. Alternatively, in some cases, illumination and imaging source can be provided on the surface of the wound dressing or housing opposite the wound facing surface.

One or more pulse oximetry (or SpO2) sensors can be used. Such sensors can measure how oxygenated the blood, pulse, or the like. To perform the measurement, pulsatile blood flow can be observed and a ratio time resolved measurement of light absorption to transmission in tissue at two different optical wavelengths can be determined. When hemoglobin in the blood becomes oxygenated, its absorption spectrum changes with regards to non-oxygenated blood. By taking a measurement at two different wavelengths, one gains a ratio metric measure of how oxygenated the blood is.

One or more pressure sensors can be used to measure pressure in or on the wound. Such pressure sensors can include differential or absolute pressure sensors. Additionally, another one or more pressure sensors can be used to measure the atmospheric pressure.

One or more motion sensors can be used to determine positioning and/or movement. The one or more motions sensors can include accelerometers, magnetometers, gyroscopes, or the like.

Additional details of sensors are disclosed in International Patent Publication No. WO2017/195038 titled “SENSOR ENABLED WOUND MONITORING AND THERAPY APPARATUS”, International Patent Application No. PCT/EP2018/059333 titled “COMPONENT STRESS RELIEF FOR SENSOR ENABLED NEGATIVE PRESSURE WOUND THERAPY DRESSINGS”, International Patent Application No. PCT/EP2018/069886 titled “SKEWING PADS FOR IMPEDANCE MEASUREMENT”, and International Patent Application No. PCT/EP2018/074200 titled “SENSOR ENABLED WOUND THERAPY DRESSINGS AND SYSTEMS IMPLEMENTING CYBERSECURITY,” the disclosure of each of which is incorporated by reference in its entirety. Any of the embodiments disclosed in these patent applications can be used with any of embodiments disclosed herein.

### Sensor Enabled Wound Dressing

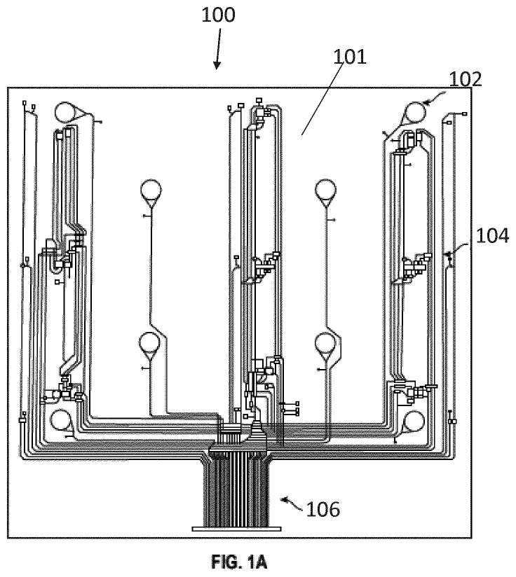

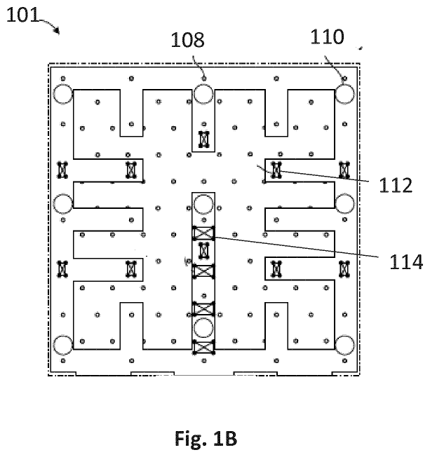

FIGS. 1A-1B show a sensor enabled wound dressing 100. The dressing 100 includes a substrate 101. The substrate 101 can include a wound contact layer on a wound facing side or the wound facing side of the substrate can be placed in or on the wound. The wound contact layer (and/or the substrate 101) can include one or more perforations for allowing wound fluid to pass through the substrate 101. The substrate 101 can incorporate a number of sensors can be utilized in order to monitor characteristics of a wound, for example, as it heals. As is illustrated, one or more sensors (and/or other electronic components) 102 connected by one or more connections 104 can be positioned or embedded in or on the substrate 101. The one or more sensors can be positioned on the wound facing side of the substrate and/or on a non-wound facing side of the substrate opposite the wound facing side.

The substrate 101 can be flexible, elastic, or stretchable or substantially flexible, elastic, or stretchable in order to conform to or cover the wound. For example, the substrate 101 can be made from a stretchable or substantially stretchable material, such as one or more of polyurethane, thermoplastic polyurethane (TPU), silicone, polycarbonate, polyethylene, polyimide, polyamide, polyester, polyethelene tetraphthalate (PET), polybutalene tetreaphthalate (PBT), polyethylene naphthalate (PEN), polyetherimide (PEI), along with various fluropolymers (FEP) and copolymers, or another suitable material. Portions of or entirety of one or more sides of the substrate 101 can be coated by a conformal coating (not shown) that can encapsulate the substrate, one or more sensors (and/or other electronic components), and/or one or more connections. Conformal coating can provide biocompatibility, shield or protect the electronics from coming into contact with fluids, or the like. Conformal coating can be flexible, elastic, or stretchable or substantially flexible, elastic, or stretchable. One or more of the sensors (and/or other electronic components) 102 and/or one or more of the electronic connections 104 can be coated with substantially non-elastic, non-flexible, non-stretchable, or rigid coating (not shown) to provide support in use when the substrate 101 becomes stretched. Additional details of the substrate and/or one or more coatings are disclosed in International Patent Application No. PCT/EP2018/059333 titled “COMPONENT STRESS RELIEF FOR SENSOR ENABLED NEGATIVE PRESSURE WOUND THERAPY DRESSINGS” and International Patent Application No. PCT/EP2018/069883 titled “BIOCOMPATIBLE ENCAPSULATION AND COMPONENT STRESS RELIEF FOR SENSOR ENABLED NEGATIVE PRESSURE WOUND THERAPY DRESSINGS,” the disclosure of each of which is incorporated by reference in its entirety. Any of the embodiments disclosed in these patent application can be used with any of embodiments disclosed herein.

Also illustrated is a connector 106 for connecting the wound dressing 100 to a controller (not shown). The controller can control at least some of the one or more sensors 102, read data collected by at least some of the one or more sensors, provide power to at least some of the one or more sensors, or the like. In some cases, the controller can be positioned in or on the substrate 101 or on another layer of the dressing 100 and connector 106 can be omitted.

The wound dressing 100 can include additional layers, such as one or more wound filler layers that can distribute negative pressure and/or absorb wound fluid, one or more fluid transport layers that can transport wound fluid through the one or more layers of the dressing (for example, vertically and/or laterally), one or more absorbent layers that can store at least some of the wound fluid, or the like. Additional details of such layers are described in International Patent Application No. PCT/EP2018/078374 titled “FLUID MANAGEMENT FOR SENSOR ENABLED WOUND THERAPY DRESSINGS AND SYSTEMS,” the disclosure of which is incorporated by reference in its entirety. Any of the embodiments disclosed in this patent application can be used with any of embodiments disclosed herein.

In some cases, the dressing 100 can include one or more antennas or transceivers for wireless communication. For example, one or more antennas or transceivers can be printed as one or more connections or traces on the substrate 101. The one or more antennas or transceivers can be used to communicate measurement data collected by the one or more sensors 102 to the controller or bypassing the controller. The one or more antennas or transceivers can be additionally or alternatively used to receive power wirelessly from a power source. In certain cases, the one or more antenna traces or transceivers can be positioned on and/or coated by substantially non-stretchable material such that the resonant frequencies of the one or more antennas or transceivers remain fixed when the substrate 101 becomes stretched due in use on a patient. Fixing the one or more resonant frequencies can be advantageous for certain communication protocols, such as RFID or the like. In some cases, a transceiver can be a separate transmitter and receiver. For example, the transceiver can include a near field communications (NFC) transmitter and a Bluetooth receiver.

One or more sensors 102 can be any of the sensors described herein. For example, one or more sensors 102 can measure one or more of impedance, SpO2, temperature, or light. For example, the illustrated sensors 102 can be conductivity sensors for measuring tissue impedance. In some cases, impedance measurement can be made utilizing a 4-point probe measurement. A drive signal, such as AC drive signal, can be generated across drive pads and the voltage measurement can be made across separate measurement pads. Measurement pads can be laid out as the corners of two concentric squares. The outer square can have approximately 80 mm side or any other suitable dimension. The inner square can have approximately 30 mm side or any other suitable dimension.

In some cases, one or more temperature sensors provide a map of the wound. The temperature sensors can be positioned equidistant with respect to each other.

In some cases, RGB sensors can be used for optical measurements. RGB sensors can incorporate one sensor at the center of the measurement area, four at a mid-distance from the center (such as, approximately 20 mm from the center) and four around the outer edges (such as, approximately 35 mm from the center). Each of the nine RGB sensors can incorporate one sensor one and one white LED.

Any distance, signal value, or the like described in the foregoing is provided for illustrative purposes. In some cases, other suitable distances, signal value, or the like can be utilized depending on the size of the measurement area, particular measurements of interest, or the like.

FIG. 1B illustrates various sensors that can be supported by the substrate 101. For example, one or more sensors, such as temperature sensors 108, conductivity sensors 110, and optical sensors 112, SpO2 sensors 114, pH sensors, or the like can be incorporated onto or into the substrate 101. The substrate 101 is illustrated as having a square shape, but it will be appreciated that the substrate may have other shapes such as rectangular, circular, oval, etc. The substrate 101 can be provided as an individual material layer that is placed over the wound area and then covered by a wound dressing apparatus or components of a wound dressing apparatus, such as gauze, foam or other wound packing material, a superabsorbent layer, a drape, a fully integrated dressing like the Pico and/or Allevyn dressing manufactured by Smith & Nephew, etc. In some cases, the substrate 101 may be part of a single unit dressing, such as described herein.

The substrate 101 can be placed in contact with the wound and allow fluid to pass through the substrate while causing little to no damage to the tissue in the wound. As described herein, the substrate can include perforations to allow fluid to pass. The substrate can include a wound contact layer can be made of a flexible material, such as silicone, and can incorporate antimicrobials or other therapeutic agents known in the art. In some cases, the substrate 101 can incorporate adhesives that adhere to wet or dry tissue. In some cases, the sensors or sensor array can be incorporated into or encapsulated within other components of the wound dressing such as the absorbent layer or spacer layer described herein.

As shown in FIG. 1B, five sensor types can be used, including, for instance, temperature sensors (such as, 25 thermistor sensors 108, in a 5×5 array, ˜20 mm pitch), oxygen saturation or SpO2 sensors 114 (such as, 4 or 5 SpO2 sensors, in a single line from the center of the wound contact layer to the edge thereof, 10 mm pitch), tissue color 112 (such as, 10 optical sensors, in 2×5 array, ˜20 mm pitch; not all 5 sensors in each row of the array need be aligned), pH (such as, by measuring color of a pH sensitive pad, optionally using the same optical sensors 112 as for tissue color), and conductivity 110 (such as, 9 conductivity sensors, in a 3×3 array, ˜40 mm pitch). As shown in FIG. 1B, the SpO2 sensors 114 can be arranged in a single line from the center of or near the center of the wound contact layer to the edge of the wound contact layer. The line of SpO2 sensors 114 can allow the sensor to take measurements in the middle of the wound, at the edge or the wound, or on intact skin to measure changes between the various regions. In some cases, the substrate 101 can be larger than the size of the wound to cover the entire surface area of the wound as well as the surrounding intact skin. The larger size of the substrate 101 and the multiple sensors can provide more information about the wound area than if the sensors were only placed in the center of the wound or in only one area at a time.

In some cases, sensors can be incorporated onto flexible circuit boards formed of flexible polymers including polyamide, polyimide (PI), polyester, polyethylene naphthalate (PEN), polyetherimide (PEI), along with various fluropolymers (FEP) and copolymers, or any material known in the art. The sensor array can be incorporated into a two-layer flexible circuit. The circuit board can be a multi-layer flexible circuit board. These flexible circuits can be incorporated into any layer of the wound dressing. For example, a flexible circuit can be incorporated into a wound contact layer. The wound contact layer can have cutouts or slits that allow for one or more sensors to protrude out of the lower surface of the wound contact layer and contact the wound area directly.

In some cases, first and second wound contact layers can be provided with the substrate 101 (such as, flexible circuit board) sandwiched between the two layers of wound contact layer material. The first wound contact layer has a lower surface intended to be in contact with the wound and an upper surface intended to be in contact with flexible circuit board. The second wound contact layer has a lower surface intended to be in contact with the flexible circuit board and an upper surface intended to be in contact with a wound dressings or one or more components forming part of an overall wound dressing apparatus. The upper surface of the first wound contact layer and the lower surface of the second wound contact layer can be adhered together with the flexible circuit board sandwiched between the two layers.

The one or more sensors can be fully encapsulated or covered by the wound contact layers to prevent contact with moisture or fluid in the wound. The first wound contact layer can have cutouts or slits that allow for one or more sensors to protrude out of the lower surface and contact the wound area directly. SpO2 sensors 114 can be mounted directly on a lower surface of the first wound contact layer. Some or all of the sensors and electrical or electronic components may be potted or encapsulated (for example, rendered waterproof or liquid-proof) with a polymer, for example, silicon or epoxy based polymers. The encapsulation with a polymer can prevent ingress of fluid and leaching of chemicals from the components. In some cases, the wound contact layer material can seal the components from water ingress and leaching of chemicals.

The components can be connected through multiple electronic connections. In some cases, temperature sensors can be arranged in groups of five. Each temperature sensor can be nominally 10 kΩ, and each group of five can have a common ground. There can be five groups of thermistors, giving a total of 30 connections. In some cases, there can be eight (as shown in FIG. 1A) or nine conductivity terminals. Each conductivity terminal can require one connection, giving a total of 8 or 9 connections. In some cases, there can be five SpO2 sensors. Each SpO2 sensor can require three connections, plus power and ground (these are covered separately), giving a total of 15 connections. In some cases, there can be 10 optical sensors. Each optical sensor can include an RGB LED and an RGB photodiode. Each optical sensor can require six connections, however five of these are common to every sensor, giving a total of 15 connections. Power and ground can be considered separately. In some cases, there can be 5 pH sensors. The pH sensors can be a color-change discs, and can be sensed using the color sensors described above. Therefore, the pH sensors require no additional connections. There can be three power rails, and seven ground return signals, giving a total of 10 common connections.

### User Activity Monitoring System

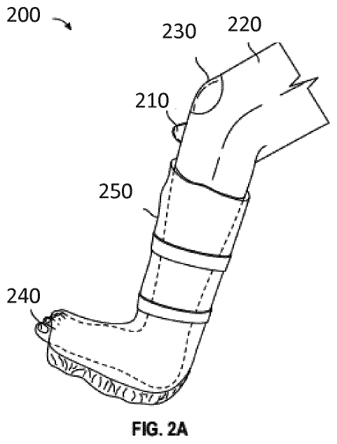

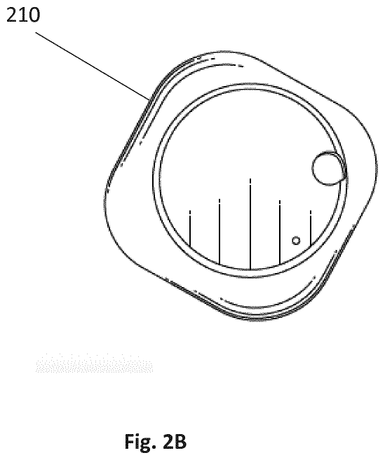

FIGS. 2A-2B show a monitoring system 200 that includes a monitoring device 210. The system 200 can monitor activity of a user. The monitored activity can include one or more of lying, standing, sitting, walking, jumping, running, squatting, or the like. Activity monitoring can be based on monitoring positioning of a body part. The monitoring device 210 can use one or more sensors as described herein. The monitoring device 210 can be attached to a body part 220. The monitoring device 210 can be attached to the body part 220 using a strap, dressing, adhesive, or other coupling mechanism and may be worn on or supported by the body.

The body part 220 can be a leg of a user that includes a knee 230 and a foot 240. As illustrated, the monitoring device 210 can be supported by the body part 220 at a position between the knee 230 and the foot 240, such as proximate to the knee 230. In some cases, the monitoring device 210 can be supported by another part of the body part 220. The monitoring device 210 can monitor and record activities (for instance, walking, jumping, sitting, laying down, running, squatting, or standing) of the body part 220, such as from a position, movement, or orientation of the monitoring device 210 or one or more other sensors of the monitoring device 210. The monitoring device 210 can, for example, be used for loading monitoring of loading of the foot 240. In certain implementations, multiple body parts can be monitored by the monitoring device 210, and different sensors can be used for monitoring different body parts.

The body part 220 is shown wearing and partly covered by an orthopedic device 130. The orthopedic device 250 can support the body part 220 and reduce a weight on the foot 240 when the user may be standing or engaging in other activities.

Although not illustrated in FIG. 2A, the user monitoring system 200 can additionally or alternatively include one or more of the monitoring device 210 at other positions, such as at a position supported by the orthopedic device 250 or another part of the body part 220. These one or more additional or alternative of the monitoring device 210 can be the same as or similar to the monitoring device 210 may monitor and record activities of the orthopedic device 250 or the another part of the body part 220.

FIG. 2B illustrates a monitoring device, such as the monitoring device 210. The monitoring device 210 can include a housing. The monitoring device 210 can be positioned in a cutout in a dressing, such as in the foam. The monitoring device 210 can include adhesive for attaching to the body part. The monitoring device 210 can include one or more motion sensors, such as accelerometers, magnetometers, gyroscopes, or the like, to measure motion data associated with the body part. The one or more sensors can be positioned on or in the housing. Motion data can be processed to determine the positioning of the body part. In some cases, the monitoring device 210 can measure one or more of pressure in a fluid flow path (which includes volume under the dressing) connecting a negative pressure source to the dressing or motion data associated with movement of the body part. In some implementations, the monitoring device 210 can measure motion data and pressure can be measured by a pressure sensor associated with the negative pressure source as described herein. The monitoring device can include a controller as described herein. The monitoring device 210 can have one or more transceivers for communicating data.

### Negative Pressure Wound Therapy System

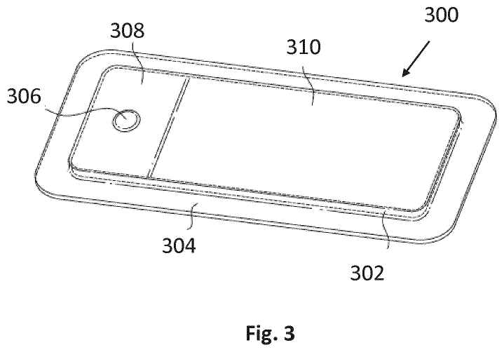

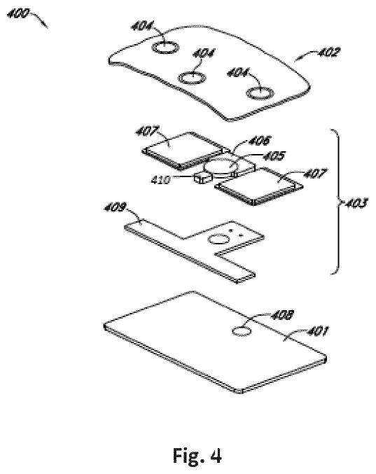

FIG. 3 shows a wound dressing 300 including one or more sensors. A negative pressure source, such as a pump, and/or other electronic components can be configured to be positioned adjacent to or next to absorbent and/or transmission layers so that the pump and/or other electronic components are part of a single article to be applied to a patient. In some cases, the pump and/or other electronics can be positioned away from the wound site. FIG. 3 illustrates a wound dressing 300 with the pump and/or other electronics positioned away from the wound site. The wound dressing can include an electronics area 308 and an absorbent area. The dressing can comprise a wound contact layer and a moisture vapor permeable film or cover layer 304 positioned above the contact layer and other layers of the dressing. The wound dressing layers and components of the electronics area as well as the absorbent area can be covered by one continuous cover layer 304. An embodiment of the electronic assembly used on dressing 300 is shown in FIG. 4.

The electronics area 308 can include a source of negative pressure (such as a pump) and some or all other components of the TNP system, such as power source(s), sensor(s), connector(s), user interface component(s) (such as button(s), switch(es), speaker(s), screen(s), etc.) and the like, that can be integral with the wound dressing. For example, the electronics area 308 can include a button or switch 306 as shown in FIG. 3A The button or switch 306 can be used for operating the pump (for example, turning the pump on/off).

The absorbent area 310 can include an absorbent material 302 and can be positioned over the wound site. The electronics area 308 can be positioned away from the wound site, such as by being located off to the side from the absorbent area. The electronics area 308 can be positioned adjacent to and in fluid communication with the absorbent area 310 as shown in FIG. 3. In some cases, each of the electronics area 308 and absorbent area 310 may be rectangular in shape and positioned adjacent to one another. The electronic components can be positioned within a recess or cut out of the absorbent material 302 but off to the side of the absorbent area.

In some cases, the electronics area 308 of the dressing can comprise electronic components with sensors as described herein. The electronics area 308 of the dressing can comprise one or more layers of transmission or spacer material and/or absorbent material and electronic components 150 can be embedded within the one or more layers of transmission or spacer material and/or absorbent material. The layers of transmission or absorbent material can have recesses or cut outs to embed the electronic components within whilst providing structure to prevent collapse. The electronic components can include a pump, power source, sensors, controller, and/or an electronics package.

A pump exhaust can be provided to exhaust air from the pump to the outside of the dressing. The pump exhaust can be in communication with the electronics area 308 and the outside of the dressing.

As used herein the upper layer, top layer, or layer above refers to a layer furthest from the surface of the skin or wound while the dressing is in use and positioned over the wound. Accordingly, the lower surface, lower layer, bottom layer, or layer below refers to the layer that is closest to the surface of the skin or wound while the dressing is in use and positioned over the wound. Additionally, the layers can have a proximal wound-facing face referring to a side or face of the layer closest to the skin or wound and a distal face referring to a side or face of the layer furthest from the skin or wound.

The electronics area 308 can include sensors or wound contact layer sensor arrays 101 as described herein below the cover layer 304 of the dressing. The electronics unit can be surrounded by a material to enclose or encapsulate a negative pressure source and electronics components by surrounding the electronics. In some cases, this material can be a casing. The electronics unit can be encapsulated or surrounded by a protective coating, for example, a hydrophobic coating as described herein. The electronics unit can be in contact with the dressing layers in the absorbent area 310 and covered by the cover layer. As used herein, the electronics unit includes a lower or wound facing surface that is closest to the wound and an opposite, upper surface, furthest from the wound when the wound dressing is placed over a wound.

In some cases, the absorbent components and electronics components can be overlapping but offset. For example, a portion of the electronics area can overlap the absorbent area, for example overlapping the superabsorber layer, but the electronics area is not completely over the absorbent area. Therefore, a portion of the electronics area can be offset from the absorbent area. The dressing layer and electronic components can be enclosed in a wound contact layer positioned below the lower most layer and a cover layer 304 positioned above the absorbent layer 302 and electronics. The wound contact layer and cover layer 304 can be sealed at a perimeter enclosing the dressing components. In some cases, the cover layer can be in direct physical contact with the absorbent material, and/or the electronics unit. In some cases, the cover layer can be sealed to a portion of the electronics unit and/or casing, for example, in areas where holes or apertures are used to accommodate the electronic components (for example, a switch and/or exhaust).

The wound dressing 300 described herein can utilize the embedded electronic assembly to generate negative pressure under the dressing. However, it can be important to protect the assembly from wound exudate or other bodily fluids that would corrode the electronics. It can also be important to protect the patient from the electric and electronic components. The electronics assembly can incorporate a pump that pull air from the dressing and exhaust to the environment in order to produce the required negative pressure differential. Therefore, it can be difficult to protect the electronics assembly and allow fluid communication between the electronic assembly and the dressing and environment surrounding the dressing. For example, complete encapsulation or potting of the assembly could prevent the movement of air from the dressing and atmosphere to the pump. As described herein, the electronic components of the electronics assembly can be protected from the environment by partial encapsulation, potting, and/or a conformable coating. In some cases, potting of electronic components can include a process of filling a complete electronic assembly with a solid or gelatinous compound for resistance to shock and vibration, exclusion of moisture, and/or exclusion of corrosive agents.

An electronics assembly can be used that includes an electronics unit positioned within an enclosure or housing, as illustrated in FIG. 4, to be incorporated into a wound dressing 300. The electronics unit can be positioned within an enclosure or housing. The housing with the electronics unit enclosed within can be placed in the dressing. FIG. 4 illustrates an electronics assembly 400 enclosing an electronics unit 403 within a housing.

As illustrated in FIG. 4, the housing of the electronics assembly 400 can include a plate 401 and flexible film 402 enclosing the electronics unit 403 within. The electronics unit 403 can include a pump 405, pump exhaust mechanism 406, power source 407, and flexible circuit board 409. The flexible film 402 can be attached to the plate 401 by welding (heat welding) or adhesive bonding to form a fluid tight seal and enclosure around the electronic components. In some cases, the flexible film 402 can be attached to the plate at a perimeter of the plate by heat welding, adhesive bonding, ultrasonic welding, RF welding, or any other attachment or bonding technique. A sensor 410 can be any of the sensors described herein. For example, the sensor 410 can be a pressure sensor configured to measure wound pressure. As another example, a second pressure sensor configured to measure atmospheric pressure can be provided in the electronics assembly 400.

The flexible film 402 can be a flexible plastic polymeric film. The flexible film 402 can be form from any material flexible polymeric film or any flexible material that confirms around the electronics. The flexible film can maintain conformability and flexibility while protecting and insulating the components within. In some cases, the flexible film 402 can be formed from a flexible or stretchable material, such as one or more of polyurethane, thermoplastic polyurethane (TPU), silicone, polycarbonate, polyethylene, methylated polyethylene, polyimide, polyamide, polyester, polyethelene tetraphthalate (PET), polybutalene tetreaphthalate (PBT), polyethylene naphthalate (PEN), polyetherimide (PEI), along with various fluropolymers (FEP) and copolymers, or another suitable material. In some cases, the flexible film 402 can be formed from polyurethane.

The plate 401 can be a plastic polymer plate. In some cases, the plate can be a flexible material to allow conformability to movement or flexing of the dressing when it is applied to a wound.

The flexible film 402 and plate 401 can be waterproof to protect the electronics unit 403 from fluid within the dressing. In some cases, the flexible film 402 can be sized appropriately so as not to limit the flexibility of the assembly. In some cases, depending on the properties of the film 402, the electronics assembly 400 can be thermoformed or vacuum formed to assist in the function of maintaining the flexibility ofthe assembly. In some cases, the electronics unit 403 can be bonded or adhered to the plate 401 within the housing such that the electronics unit 403 cannot move within.

In some cases, the housing can include one or more windows 404. The windows 404 can be a porous film or membrane that can allow gas to pass through. The windows 404 can be a hydrophobic film or membrane. In some cases, the hydrophobic nature of the window 404 can repel wound fluids and prevent the leak of fluids into the electronics assembly 400. In some cases, the windows 404 can include a bacterial filter. In some cases, the windows 404 can have the porosity that enables them to act as a bacterial filter and preventing bacterial release from the body fluids into the environment. The windows 404 can also prevent the ingress of bacteria from the environment to the wound site.

The electronics assembly 400 can have more than one windows 404 or a larger window 404 to provide a sufficiently large area for air movement therethrough, thus minimizing the pressure drop across the membrane and hence the power consumption of the system in achieving the pressure differential. In some cases, the electronics assembly 400 can include several windows with a small area. In other cases, the electronics assembly can include a window with a single large area.

The electronics assembly 400 illustrated in FIG. 4 can be incorporated within the wound dressing such that, once the dressing is applied to the body of the patient, air from within the dressing can pass through the windows 404 to be pumped out in the direction shown by the arrow on the pump 405. The exhausted air from the pump can pass out of the pump assembly through the pump exhaust mechanism 406 and be exhausted or vented from the housing of the electronics assembly 400 through an aperture or vent 408 in the plate 401. In some cases, the flexible circuit board 409 can be positioned between the exhaust mechanism 406 and the plate 401. The flexible circuit board 409 can also include an aperture or vent aligned with the exhaust hole in the exhaust mechanism. The vent hole or apertures in the exhaust mechanism 406, flexible circuit board 409, and plate 401 can be aligned and sealed to each other. This seal can ensure the pump exhaust is exhausted from the electronics assembly 400 through the vent 408 in the plate 401. In other cases, the exhaust mechanism 406 of the electronics unit 403 can be positioned on and bonded directly to the plate 401 with an airtight seal.

The electronics assembly 400 can be embedded within the wound dressing in the same manner as the electronics unit described with reference to FIG. 3. The dressing can have one or more absorbent layers within the dressing and the absorbent layers can have a single aperture or recess for receiving the electronics assembly within. In some cases, the electronics assembly can be positioned below the overlay layer similar to the electronics unit. In such cases, the overlay layer would include an aperture to allow access to at least a portion of the top surface of the plate 401.

When the electronics assembly 400 is positioned within the dressing it can be positioned below the wound cover and the overlay layer. In other cases, an overlay layer is not used and the electronics assembly 400 is positioned directly below the cover layer or backing layer.

The cover layer or backing layer can include an aperture exposing a portion of, most of, or all of the top surface of the plate 401. The aperture in the cover layer can be positioned over at least a portion of the plate 401 to allow access to at least a portion of the plate 401 positioned below the cover layer. In some cases, the cover layer can have a plurality of apertures over the top surface of the plate 401. For example, the cover layer can have apertures over the vent holes, indicator portions, and/or switch cover. In other cases, the cover layer can have a single aperture over the top surface of the plate 401 including but not limited to the vent holes, indicator portions, and/or switch cover.

### Control System

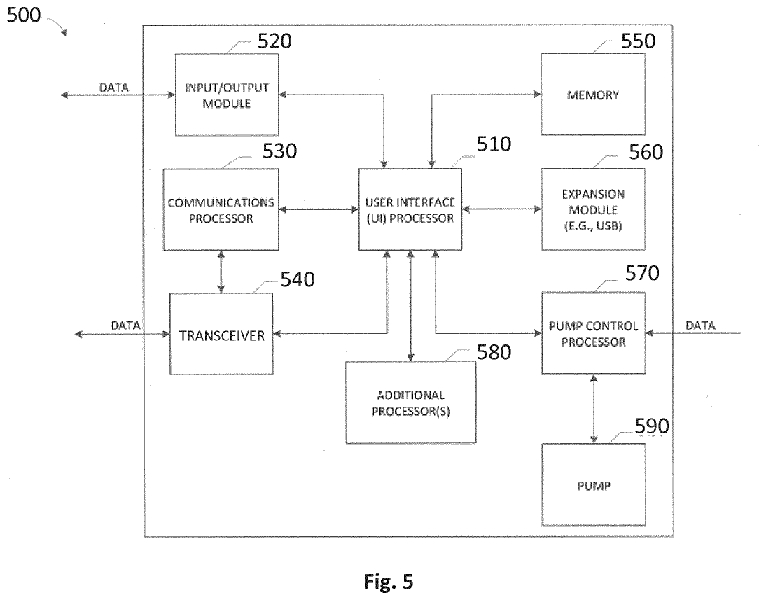

FIG. 5 illustrates a schematic of a control system 500 which can be employed in any of the embodiments of wound monitoring and/or treatment systems described herein. Electrical components can operate to accept user input, provide output to the user, operate the negative pressure source of a TNP system, provide network connectivity, and so on. It may be advantageous to utilize multiple processors in order to allocate or assign various tasks to different processors. In some cases, a first processor can be responsible for user activity and a second processor can be responsible for controlling another device, such as a pump 590. This way, the activity of controlling the other device, such as the pump 590, which may necessitate a higher level of responsiveness (corresponding to higher risk level), can be offloaded to a dedicated processor and, thereby, will not be interrupted by user interface tasks, which may take longer to complete because of interactions with the user.

Input and output to the other device, such as a pump 590, one or more sensors (as described herein), or the like, can be controlled by an input/output (I/O) module 520. For example, the I/O module can receive data from one or more sensors through one or more ports, such as serial (for example, 12C), parallel, hybrid ports, and the like. The processor 510 also receives data from and provides data to one or more expansion modules 560, such as one or more USB ports, SD ports, Compact Disc (CD) drives, DVD drives, FireWire ports, Thunderbolt ports, PCI Express ports, and the like. The processor 510, along with other controllers or processors, stores data in one or more memory modules 550, which can be internal and/or external to the processor 510. Any suitable type of memory can be used, including volatile and/or non-volatile memory, such as RAM, ROM, magnetic memory, solid-state memory, Magnetoresistive random-access memory (MRAM), and the like.

In some cases, the processor 510 can be a general purpose controller, such as a low-power processor. In other cases, the processor 510 can be an application specific processor. In some cases, the processor 510 can be configured as a “central” processor in the electronic architecture of the system 500, and the processor 510 can coordinate the activity of other processors, such as a pump control processor 570, communications processor 530, and one or more additional processors 580. The processor 510 can run a suitable operating system, such as a Linux, Windows CE, VxWorks, etc.

The pump control processor 570 (if present) can be configured to control the operation of a negative pressure pump 590 (if present). The pump 590 can be a suitable pump, such as a diaphragm pump, peristaltic pump, rotary pump, rotary vane pump, scroll pump, screw pump, liquid ring pump, diaphragm pump operated by a piezoelectric transducer, voice coil pump, and the like. In some cases, the pump control processor 570 can measure pressure in a fluid flow path, using data received from one or more pressure sensors, calculate the rate of fluid flow, and control the pump. In some cases, the pump control processor 570 controls the pump motor so that a desired level of negative pressure in achieved in the wound cavity 110. The desired level of negative pressure can be pressure set or selected by the user. The pump control processor 570 can control the pump (for example, pump motor) using pulse-width modulation (PWM). A control signal for driving the pump can be a 0-100% duty cycle PWM signal. The pump control processor 570 can perform flow rate calculations and detect alarms. The pump control processor 570 can communicate information to the processor 510. The pump control processor 570 can include internal memory and/or can utilize memory 550. The pump control processor 570 can be a low-power processor.

A communications processor 530 can be configured to provide wired and/or wireless connectivity. The communications processor 530 can utilize one or more antennas or transceivers 540 for sending and receiving data. In some cases, the communications processor 530 can provide one or more of the following types of connections: Global Positioning System (GPS) technology, cellular connectivity (for example, 2G, 3G, LTE, 4G, 5G, or the like), WiFi connectivity, Internet connectivity, and the like. Connectivity can be used for various activities, such as pump assembly location tracking, asset tracking, compliance monitoring, remote selection, uploading of logs, alarms, and other operational data, and adjustment of therapy settings, upgrading of software and/or firmware, and the like. In some cases, the communications processor 530 can provide dual GPS/cellular functionality. Cellular functionality can, for example, be 3G functionality. In such cases, if the GPS module is not be able to establish satellite connection due to various factors including atmospheric conditions, building or terrain interference, satellite geometry, and so on, the device location can be determined using the 3G network connection, such as by using cell identification, triangulation, forward link timing, and the like. In some cases, the system 500 can include a SIM card, and SIM-based positional information can be obtained.

The communications processor 530 can communicate information to the processor 510. The communications processor 530 can include internal memory and/or can utilize memory 550. The communications processor 530 can be a low-power processor.

In some cases, the system 500 can store data illustrated in Table 1. This data can be stored, for example, in memory 550. This data can include patient data collected by one or more sensors. In various cases, different or additional data can be stored by system 500. In some cases, location information can be acquired by GPS or any other suitable method, such as cellular triangulation, cell identification forward link timing, and the like.

TABLE 1
Example Data Stored

| Category | Item | Type | Source |
|---|---|---|---|
| GPS | Location | Latitude, Longitude, | Acquired from GPS |
| | | Altitude | |
| | Timestamp Location Acquired | Timestamp | |
| Therapy | Total time therapy ON since device | Minutes | Calculated on device |
| | activation | | based on user control |
| | Total time therapy ON since last | Minutes | |
| | maintenance reset | | |
| | Device Placement; accumulated daily | Minutes | |
| | hours starting from first Therapy ON | | |
| | after last maintenance reset, stopping | | |
| | at last Therapy OFF before returning | | |
| | for Maintenance and maintenance | | |
| | reset. (Includes both THERAPY ON and | | |
| | THERAPY OFF hours) | | |
| Device | Serial Number | Alphanumeric | Set by Pump Utility |
| | Controller Firmware Version | Alphanumeric | Unique version |
| | | | identifier, hard coded in |
| | | | firmware |
| Events | Device Event Log (See Table 3 | List of Events | Generated in response to |
| | for example) | (See Table 2) | various user actions and |
| | | | detected events |

The system 500 can track and log therapy and other operational data. Such data can be stored, for example, in the memory 550. In some cases, the system 500 can store log data illustrated in Table 2. Table 3 illustrates an example event log. One or more such event logs can be stored by the system 500. As is illustrated, the event log can include time stamps indicating the time of occurrence. In some cases, additional and/or alternative data can be logged.

TABLE 2
Example Data Tracked

| Category | ID | Type | Data Content | Notes |
|---|---|---|---|---|
| Device | 0 | Startup (Created DB) | | First time, out-of-the-box. |
| | 1 | Startup (Resumed DB) | | Subsequent power-ups. |
| | 2 | Startup (Corrupt DB, Recreated) | | Corrupt configuration was detected. The |
| | | | | database was deleted and recreated, and |
| | | | | next ran was in out-of-the-box mode. |
| | 3 | Shutdown (Signaled) | | Normal shutdown, handled/registered by |
| | | | | software. |
| | 4 | Shutdown (Inferred) | | Unexpected shutdown; on next power-up, |
| | | | | last active time registered as shutdown |
| | | | | event. |
| Therapy | 5 | Start Delivery (Continuous) | modes, setpoints | Modes are Y-connect status, and intensity. |
| | 6 | Start Delivery (Intermittent) | modes, setpoints | Modes are Y-connect status, and intensity. |
| | 7 | Stop Delivery | | |
| | 8 | Set Therapy Pressure Setpoint | mmHg | This and other therapy adjustment events |
| | | | | are only recorded while therapy is being |
| | | | | delivered. |
| | 9 | Set Standby Pressure Setpoint | mmHg | |
| | 10 | Set Intermittent Therapy Duration | setting (30 s, 60 s, etc) | |
| | 11 | Set Intermittent Standby Duration | setting (30 s, 60 s, etc) | |
| | 12 | SetMode | cont/intermittent | |
| | 13 | Set Intensity | low/med/high | |
| | 14 | Set Y Connect | yes/no | |
| Alarm | 15 | Over Vacuum | high mmHg | |
| | 16 | High Vacuum | high deviation mmHg | |
| | 17 | Blocked Full Canister | low airflow 1 pm | |
| | 18 | High Row Leak | high airflow 1 pm | |
| | 19 | Low Vacuum | low mmHg | |
| | 20 | Battery Failure | | |
| | 21 | Critical Battery | | |
| | 22 | Low Battery | | |
| | 23 | Inactivity | | |
| Maintenance | 24 | Maintenance Reset | | |
| | 25 | Reset to Defaults | | |
| | 26 | Software/Device Warning | Warning code | Any detected, minor unexpected software |
| | | | | behavior will be logged as an event |
| | 27 | Software/Device Fault | Fault code | Any detected, severe unexpected software |
| | | | | behavior will be logged as an event |

TABLE 3
Example Event Log

| | Type | | |
|---|---|---|---|
| Timestamp | ID | Type Description | Data |
| 1:23:45 4/2/2012 (UTC-12) | 0 | Startup (Created DB) | |
| 1:29:23 4/2/2012 (UTC-12) | 15 | Set Intensity | medium |
| 1:29:43 4/2/2012 (UTC-12) | 10 | Set Therapy Pressure Setpoint | 120 mmHg |
| 1:31:02 4/2/2012 (UTC-12) | 7 | Start Delivery (Continuous) | 120 mmHg continuous, |
| | | | medium intensity, no Y |
| | | | connect |
| 1:44:20 4/2/2012 (UTC-12) | 20 | High Flow Leak | 4l pm |
| 1:44:24 4/2/2012 (UTC 12) | 9 | Stop Delivery | |

In some cases, using the connectivity provided by the communications processor 530, the system 500 can upload any of the data stored, maintained, and/or tracked by the system 500 to a remote computing device. In some cases, the following information can be uploaded to the remote computing device: activity log(s), which includes therapy delivery information, such as therapy duration, alarm log(s), which includes alarm type and time of occurrence; error log, which includes internal error information, transmission errors, and the like; therapy duration information, which can be computed hourly, daily, and the like; total therapy time, which includes therapy duration from first applying a particular therapy program or programs; lifetime therapy information; device information, such as the serial number, software version, battery level, etc.; device location information; patient information; and so on. The system 500 can also download various operational data, such as therapy selection and parameters, firmware and software patches and upgrades, and the like. The system 500 can provide Internet browsing functionality using one or more browser programs, mail programs, application software (for example, apps), etc Additional processors 580, such as processor for controlling one or more user interfaces (such as, one or more displays), can be utilized. In some cases, any of the illustrated and/or described components of the system 500 can be omitted depending on an embodiment of a wound monitoring and/or treatment system in which the system 500 is used.

### Authorization of Data Acquisition

As described herein, embodiments of the wound monitoring and/or treatments systems can collect patient data using the one or more sensors. Collected patient data can be stored in a memory of the system (such as, the memory 550) and/or transmitted to a remote computing system. In some cases, data privacy and security regulations, such as HIPPA in the United States, GDPR in Europe, or the like, can dictate that a previous authorization of collection of at least some types of patient data be given prior to initiating the collection. Such authorization can be given by an authorized user, for example, a health care professional (HCP), such as a physician, nurse, or the like.

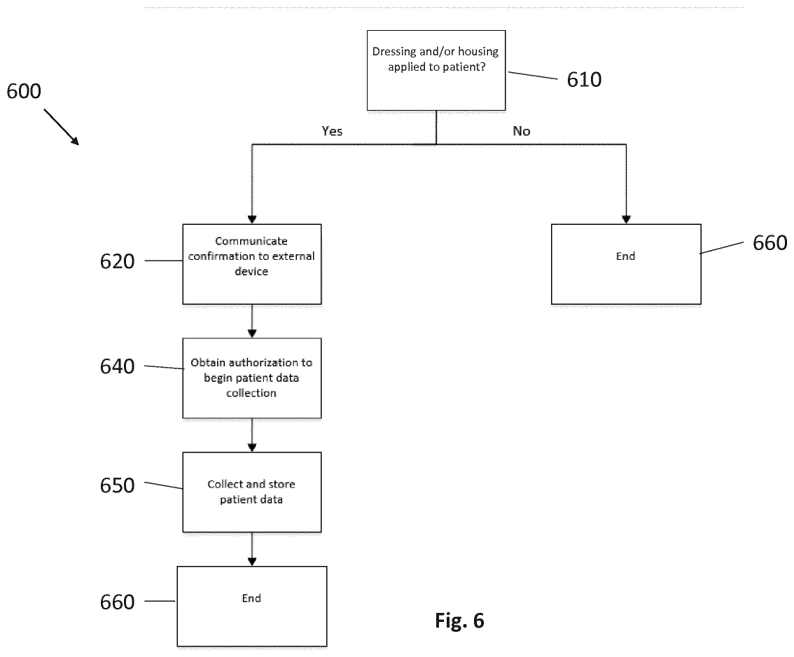

FIG. 6 illustrates a flow chart of a process 600 for authorizing collection of patient data. The process 600 can be implemented by any of the systems disclosed herein, including by any of the controllers disclosed herein. In block 610, the process 600 can determine that the dressing and/or housing supporting one or more sensors is positioned on or applied to the patient. This determination can be performed using data collected by the one or more sensors and/or by receiving a confirmation from a user, such as HCP (for example, via a button press).

For example, the process 600 can determine that the dressing and/or housing is positioned on or applied to the patient in response to verifying that temperature measured by one or more temperature sensors of the one or more sensors is within a physiological temperature range, such as between about 35 and 41 degrees Celsius (or about 95 and 105.8 degrees Fahrenheit). Additionally or alternatively, the process 600 can determine a difference between the temperature measured by a temperature sensor and ambient temperature measured by an ambient temperature sensor, and based on the difference satisfying one or more thresholds, determine that the dressing and/or housing is positioned on or applied to the patient. The one or more thresholds can be associated with a temperature difference between, for example, room temperature (between about 15 and 25 degrees Celsius or about 59 and 77 degrees Fahrenheit) and physiological temperature described herein. In some cases, the ambient temperature sensor can be positioned in or on the orthopedic device 250 (see FIG. 2A).

As another example, the process 600 can determine that the dressing and/or housing is positioned on or applied to the patient in response to verifying that pressure measured by one or more pressure sensors of the one or more sensors is indicative of expected pressure associated with a height at which the dressing and/or housing is expected to be positioned. For instance, with reference to FIG. 2A, the device 210 can be configured for positioning at a particular location on the body part 220 (such as below the knee but above the foot 240), and such location can be associated with an expected pressure. Additionally or alternatively, the process 600 can determine a difference between the pressure measured by the pressure sensor and atmospheric pressure measured by an atmospheric pressure sensor in order to determine if the pressure difference satisfies a threshold indicative of the expected pressure. This may be advantageous because the expected pressure can vary at different altitudes. In some cases, a single pressure sensor that measures pressure relative to absolute pressure can be used, and the threshold indicative of the expected pressure can be fixed regardless of the altitude.

As another example, the process 600 can determine that the dressing and/or housing is positioned on or applied to the patient in response to verifying that pressure measured by the pressure sensor matches a pressure profile of a negative pressure wound therapy being applied to a wound. In some cases, changes in magnitude (and/or frequency) of the pressure over time can be indicative of a steady state condition during application of negative pressure wound therapy to a wound. Such changes can be compared to one or more thresholds to determine if the changes in the magnitude (and/or frequency) of pressure are indicative of the steady state condition. This can be distinguished from chaotic changes in the pressure resulting from a negative pressure system not being coupled to a wound. Additional details of determining that a negative pressure system is coupled to a wound are disclosed in International Patent Publication No. WO2017/197357 titled “AUTOMATIC WOUND COUPLING DETECTION IN NEGATIVE PRESSURE WOUND THERAPY SYSTEMS,” the entire disclosure of which is incorporated by reference in its entirety. Any of the embodiments disclosed in this patent application can be used with any of embodiments disclosed herein.

As another example, the process 600 can determine that the dressing and/or housing is positioned on or applied to the patient in response to verifying that impedance measured by one or more conductivity sensors of the one or more sensors is indicative of impedance variations of living tissue. In some cases, flow of wound fluid can cause the impedance to vary over time. In response to comparing such variation(s) determined by the one or more conductivity sensors to one or more thresholds, it can be determined that the dressing and/or housing is positioned on or applied to the patient. The one or more thresholds can be selected to distinguish impedance changes of living tissue from substantially constant impedance of a non-living matter (such as, table, shelf, or the like). For instance, the one or more thresholds can be selected to distinguish a non-zero value indicative of variations associated with tissue from zero.

As another example, the process 600 can determine that the dressing and/or housing is positioned on or applied to the patient in response to verifying that image data obtained by one or more image sensors of the one or more sensors is indicative of image data of living tissue. In some cases, flow of wound fluid can cause wound color to vary over time. In response to comparing such variation(s) determined by the one or more image sensors to one or more thresholds, it can be determined that the dressing and/or housing is positioned on or applied to the patient. The one or more thresholds can be selected to distinguish color changes of living tissue from substantially non-varying color of a non-living matter (such as, table, shelf, or the like). For instance, the one or more thresholds can be selected to distinguish a non-zero value indicative of variations associated with tissue from zero.

As another example, the process 600 can determine that the dressing and/or housing is positioned on or applied to the patient in response to verifying that image data obtained by one or more blood oxygen saturation sensors of the one or more sensors is indicative of physiological levels, such as oxygen saturation between about 90% to 100%, pulse between about 30 to 200 beats per minute, or the like. In response to comparing blood oxygen saturation (or pulse) determined by the one or more blood oxygen saturation sensors to one or more thresholds, it can be determined that the dressing and/or housing is positioned on or applied to the patient.

As another example, the process 600 can determine that the dressing and/or housing is positioned on or applied to the patient in response to verifying that image data obtained by one or more pH sensors of the one or more sensors is indicative of physiological levels, such as between about 7.2 to 7.4. In response to comparing pH determined by the one or more pH sensors to one or more thresholds, it can be determined that the dressing and/or housing is positioned on or applied to the patient.

Addition or alternatively, flow of wound fluid can cause pH levels to vary over time. In response to comparing such variation(s) determined by the one or more pH sensors to one or more thresholds, it can be determined that the dressing and/or housing is positioned on or applied to the patient. The one or more thresholds can be selected to distinguish pH changes of living tissue from substantially non-varying pH of a non-living matter (such as, table, shelf, or the like). For instance, the one or more thresholds can be selected to distinguish a non-zero value indicative of variations associated with tissue from zero.

As another example, the process 600 can determine that the dressing and/or housing is positioned on or applied to the patient in response to verifying that motion data obtained by one or more motion sensors of the one or more sensors is indicative of activity of a patient. In response to comparing activity determined by the one or more motion sensors to one or more thresholds, it can be determined that the dressing and/or housing is positioned on or applied to the patient. For instance, a patient can be requested to engage in an activity or a series of activities (for example, walk, sit, squat, jump, run, etc.) after the dressing and/or housing has been positioned on the patient and this activity can be detected and compared to one or more thresholds indicative of the activity. For instance, the one or more thresholds can be selected to distinguish a non-zero value indicative of activity from zero.

If the process 600 determines that the dressing and/or housing is not positioned on or applied to the patient, the process can transition to block 660 and ends. Otherwise, the process 600 can transition to block 620. At block 620, the process 600 can confirm that the dressing and/or housing is positioned on or applied to the patient to a remote or an external computing device, such as a remote server. For example, HCP can pair the dressing and/or housing with an external user device, such as a smartphone, tablet, computer, etc., and confirmation can be transmitted to the external computing device. The confirmation can be transmitted over a network.

At block 640, the process 600 can obtain authorization to begin patient data collection by one or more sensors of the dressing and/or housing. In some cases, the HCP can authorize collection of patient data by, for example, sending an authorization from the external computing device. In some cases, the HCP can authorize data collection via a user interface of the dressing and/or housing. For example, authorization can be provided via a physical input, such as a button press, removal of a cover exposing a light sensor, or the like. The HCP can verify if his or her authorization has been communicated via feedback, such as user interface feedback or feedback received from by the external computing device. User interface feedback can be provided visually, audibly, tactilely, or the like. The authorization can serve as and/or include a time stamp indicating start of a patient data collection episode. The time stamps can indicate the start of a patient data collection period and/or the end of a patient data collection period. In some cases, the authorization (for example, the time stamp) can additionally or alternatively include HCP identity reference data or biometric identity data of the HCP. In some cases, the authorization (for example, the time stamp) can additionally or alternatively include confirmation of patient location and/or type of treatment (for example, monitoring and/or therapy).

In some cases, if the authorization is not received in block 640, the process 600 can prevent storing at least some collected patient data. Certain patient data can be stored without authorization. Such data can include therapy time, location information, time that system is powered on, certain motion data, or the like.

At block 650, the process 600 can collect and/or store patient data obtained by the one or more sensors. The process 600 can store at least some of collected patient data in memory as described herein.

In some cases, the process 600 can continue collecting and storing patient data until receiving an indication to stop. The indication can be received from the external computing device and/or via the user interface of the dressing and/or housing. The indication can be provided by the HCP. The process 600 can transition to block 660 where it can prevent storing of at least some patient data collected subsequent to receiving the indication. The indication can include a time stamp indicating an end of the patient data collection episode. The process 600 can use the time stamps indicating the beginning and end of the patient data collection episode to associate collected and/or stored patient data with the patient data collection episode.

In some cases, collected and/or stored patient data can be used to troubleshoot and verify the correct attachment of the one or more sensors. For example, the HCP can use the data and ask the user to move or walk around to verify that the one or more sensors are working (for example, collecting valid data). This can be particularly useful with the system 200 as described herein.

In some cases, collected and/or stored patient data can be sent to an external computing device to create a patient device object. Patient data can be measured, processed, and be securely protected and transmitted, for example, to a remote “medical” cloud in compliance with patient privacy acts in real time of after data acquisition is completed. For example, patient data can be transmitted after completion of the patient data collection episode. Subsequent patient data or vital signs acquisition could follow the same architecture and processes.

In some cases, initiation of data collection can be indicated to the HCP using any of the methods described herein. For example, indication can be in the form of one or more alerts or alarms. In case of one or more wireless sensors, the alerts or alarms can be provided on a wirelessly paired device, such as a tablet, phone, computer. In some cases, one or more additional alerts or alarms can be provided. For instance, an alarm or alert can be generated when one or more pressure sensors indicate high pressure on certain wound areas or lack of pressure may signal that the user is not following the treatment plan (such as, not complying with physical therapy routines). This can signal to the HCP that the patient is not following the HCP's directions. The HCP can then respond by contacting the user to abide by the HCP's directions.

In some cases, data collection can help future data analysis to determine, for example, compliance with prescribed therapy. This data can be better used to monitor and/or treat patients. Patient data can allow manufactures, HCPs, and other users to analyze and improve future sensor attachment and/or placement processes. After proper authorization and confirmation of proper sensor placement and system operation, the HCP can begin the real patient behavioural data collection process. The HCP can also verify if the process has started via receiving an alarm or alert (for example, on a tablet, smart phone, phone, remote portal, etc.). Once the data collection begins, the HCP can allow the user to leave. While the user is at a remote location, such as home, the system can still send data which the HCP can monitor. When the data acquisition is complete, the HCP can end completely or momentarily the data collection as described herein.

For example, in connection with the system in 200, the process 600 can be implemented as follows. The activity monitoring device 210 can be attached to the patient. The device 210 can be paired with an external computing device, such as a phone, tablet, or the like. The pairing can be wireless or wired. Pairing can provide for security of patient data. For example, the device 210 can transmit patient data only to a the paired external computing device, which in turn can transmit the data to another external computing device, such as a cloud server. Subsequent to the pairing, patient data can be used to verify correct attachment of the device 210. In some cases, another activity monitoring device similar to the device 210 can be attached to or incorporated in the orthopaedic device 250, and correct positioning of the device 250 can be verified. For example, patient data can include motion data, and correct positioning of one or more the device 210 or device 250 can be verified as described herein.

Patient data can be sent to the cloud server to create one or more new patient device objects. Such one or more objects can indicate readiness for authorization of patient data collection. In some cases, patient data described in the preceding paragraph can be of type that does not require authorization as described herein.

Subsequent to verification of correct positioning, HCP can provide authorization for patient data collection as described herein. HCP can verify that data collection has been started via an alarm or alert as described herein. Collected data can be transmitted to the cloud server. Cloud server can add the data to the one or more patient device objects. At a future time, HCP can pause or stop data collection as described herein.

In some cases, patient data can be used to determine that the patient is complying with orthopaedic therapy by verifying that the device 250 is being worn. This can be accomplished by, for example, comparing the motion data collected by the device 250 and determining that it matches the motion data collected by the device 210.

### Terminology

Many other variations than those described herein will be apparent from this disclosure. For example, depending on the embodiment, certain acts, events, or functions of any of the steps described herein can be performed in a different sequence, can be added, merged, or left out altogether (e.g., not all described acts or events are necessary for the practice of the algorithms). Moreover, in certain embodiments, acts or events can be performed concurrently. In addition, different tasks or processes can be performed by different machines and/or computing systems that can function together.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figure. Depending on the embodiment, certain of the steps described above may be removed, others may be added. For instance, the various components illustrated in the figures may be implemented as software or firmware on a processor, controller, ASIC, FPGA, or dedicated hardware. Hardware components, such as processors, ASICs, FPGAs, and the like, can include logic circuitry. Furthermore, the features and attributes of the specific embodiments disclosed above may be combined in different ways to form additional embodiments, all of which fall within the scope of the present disclosure.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the described embodiments, and may be defined by claims as presented herein or as presented in the future.

Conditional language used herein, such as, among others, “can,” “could”, “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

The scope of the present disclosure is not intended to be limited by the description of certain embodiments and may be defined by the claims. The language of the claims is to be interpreted broadly based on the language employed in the claims and not limited to the examples described in the present specification or during the prosecution of the application, which examples are to be construed as non-exclusive.

1. A wound monitoring and/or treatment system comprising:
a dressing, housing, or dressing and housing configured to be placed in or over a wound, skin, or wound and skin of a patient;
a sensor positioned on or in the dressing, housing, or dressing and housing and configured to measure patient data, the sensor comprising at least one of a pressure sensor, conductivity sensor, blood oxygen saturation sensor, optical sensor, pH sensor, temperature sensor, or motion sensor;
a transceiver; and
a controller configured to receive patient data measured by the sensor, selectively store at least some of the patient data in a memory, and communicate, via the transceiver, at least some of the patient data stored in the memory to an external computing device, the controller further configured to:
determine based on the patient data measured by the sensor if the dressing, housing, or dressing and housing is placed in or over the wound, skin, or wound and skin of the patient;
in response to determining that the dressing, housing, or dressing and housing is placed in or over the wound, skin, or wound and skin of the patient, communicate, via the transceiver, to the external computing device a confirmation that the dressing, housing, or dressing and housing is placed in or over the wound, skin, or wound and skin of the patient and causing the external computing device to receive an authorization from a healthcare provider (HCP) to collect the patient data; and
in response to receiving, via the transceiver, from the external computing device the authorization to collect patient data, store at least some of the patient data measured by the sensor in the memory.

2. The system of claim 1, wherein the controller is further configured to, in response to not receiving the authorization to collect patient data, prevent storing at least some of the patient data in the memory.

3. The system of claim 1, wherein the sensor comprises a temperature sensor and the controller is configured to determine that the dressing, housing, or dressing and housing is placed in or over the wound, skin, or wound and skin of the patient in response to determining that patient temperature measured by the temperature sensor is within a temperature range.

4. The system of claim 3, further comprising an ambient temperature sensor positioned on or in the dressing, housing, or dressing and housing and configured to measure an ambient temperature, wherein the controller is configured to determine that the dressing, housing, or dressing and housing is placed in or over the wound, skin, or wound and skin of the patient in response to determining that a difference between temperature measured by the sensor and the ambient temperature satisfies a temperature difference threshold.

5. The system of claim 1, wherein the sensor comprises a pressure sensor and the controller is configured to determine that the dressing, housing, or dressing and housing is placed in or over the wound, skin, or wound and skin of the patient in response to determining that pressure measured by the pressure sensor satisfies a pressure threshold.

6. The system of claim 5, further comprising an ambient pressure sensor positioned on or in the dressing, housing, or dressing and housing and configured to measure an ambient pressure, wherein the controller is configured to determine that the dressing, housing, or dressing and housing is placed in or over the wound, skin, or wound and skin of the patient in response to determining that a difference between pressure measured by the pressure sensor and the ambient pressure satisfies a pressure difference threshold.

7. The system of claim 1, wherein the sensor comprises a conductivity sensor and the controller is configured to determine that the dressing, housing, or dressing and housing is placed in or over the wound, skin, or wound and skin of the patient in response to determining that conductivity measured by the conductivity sensor satisfies a conductivity threshold.

8. The system of claim 1, wherein the sensor comprises an optical sensor and the controller is configured to determine that the dressing, housing, or dressing and housing is placed in or over the wound, skin, or wound and skin of the patient in response to determining that image data measured by the optical sensor is associated with image data of a wound, skin, or wound and skin.

9. The system of claim 1, wherein the sensor comprises a blood oxygen saturation sensor and the controller is configured to determine that the dressing, housing, or dressing and housing is placed in or over the wound, skin, or wound and skin of the patient in response to determining that blood oxygen saturation measured by the blood oxygen saturation sensor is within blood oxygen saturation range.

10. The system of claim 1, wherein the sensor comprises a pH sensor and the controller is configured to determine that the dressing, housing, or dressing and housing is placed in or over the wound, skin, or wound and skin of the patient in response to determining that pH level measured by the pH sensor satisfies a pH threshold.

11. The system of claim 1, wherein the sensor comprises a motion sensor and the controller is configured to determine that the dressing, housing, or dressing and housing is placed in or over the wound, skin, or wound and skin of the patient in response to determining that motion data measured by the motion sensor satisfies a motion threshold.

12. The system of claim 1, wherein the controller is further configured to:
receive, via the transceiver, from the external computing device an indication by the HCP to stop patient data collection; and
in response to receiving the indication, prevent storing in memory at least some of the patient data received from the sensor subsequent to the indication.

13. The system of claim 12, wherein the authorization includes a first time stamp indicating a start of a patient data collection episode, wherein the indication includes a second time stamp indicating an end of the patient data collection episode, and wherein the controller is further configured to associate patient data stored after receiving the first time stamp and before receiving the second time stamp as being associated with the patient data collection episode.

14. (canceled)

15. A method of authorizing collection of patient data comprising:
by a controller configured to be in communication with a dressing, housing, or dressing and housing configured to be placed in or over a wound, skin, or wound and skin of a patient, wherein a sensor is positioned on or in the dressing, housing, or dressing and housing, and wherein the sensor is configured to measure patient data:
determining based on the patient data measured by the sensor if the dressing, housing, or dressing and housing is placed in or over the wound, skin, or wound and skin of the patient;
in response to determining that the dressing, housing, or dressing and housing is placed in or over the wound, skin, or wound and skin of the patient, communicating, via a transceiver in communication with the controller, to an external computing device a confirmation that the dressing, housing, or dressing and housing is placed in or over the wound, skin, or wound and skin of the patient and causing the external computing device to receive an authorization from a healthcare provider (HCP) to collect the patient data; and
in response to receiving, via the transceiver, from the external computing device the authorization to collect the patient data, storing at least some of the patient data measured by the sensor in a memory.

16. The method of claim 15, further comprising by the controller, in response to not receiving the authorization to collect patient data, preventing storing at least some of the patient data in the memory.

17. The method of claim 15, wherein an ambient temperature sensor is positioned on or in the dressing, housing, or dressing and housing and is configured to measure an ambient temperature, wherein the sensor comprises a temperature sensor, and wherein the method comprises determining that the dressing, housing, or dressing and housing is placed in or over the wound, skin, or wound and skin of the patient in response to determining a difference between temperature measured by the sensor and the ambient temperature satisfies a temperature difference threshold.

18. (canceled)

19. The method of claim 15, wherein an ambient pressure sensor is positioned on or in the dressing, housing, or dressing and housing and is configured to measure an ambient pressure, wherein the sensor comprises a pressure sensor, and wherein the method comprises determining that the dressing, housing, or dressing and housing is placed in or over the wound, skin, or wound and skin of the patient in response to determining that a difference between pressure measured by the pressure sensor and the ambient pressure satisfies a pressure difference threshold.

20. (canceled)

21. The method of claim 15, wherein:
the sensor comprises a conductivity sensor and the method comprises determining that the dressing, housing, or dressing and housing is placed in or over the wound, skin, or wound and skin of the patient in response to determining that conductivity measured by the conductivity sensor satisfies a conductivity threshold; or
wherein the sensor comprises an optical sensor and the method comprises determining that the dressing, housing, or dressing and housing is placed in or over the wound, skin, or wound and skin of the patient in response to determining that image data measured by the optical sensor is associated with image data of a wound, skin, or wound and skin; or
wherein the sensor comprises a blood oxygen saturation sensor and the method comprises determining that the dressing, housing, or dressing and housing is placed in or over the wound, skin, or wound and skin of the patient in response to determining that blood oxygen saturation measured by the blood oxygen saturation sensor is within blood oxygen saturation range; or
wherein the sensor comprises a pH sensor and the method comprises determining that the dressing, housing, or dressing and housing is placed in or over the wound, skin, or wound and skin of the patient in response to determining that pH level measured by the pH sensor satisfies a pH threshold; or
wherein the sensor comprises a motion sensor and the method comprises determining that the dressing, housing, or dressing and housing is placed in or over the wound, skin, or wound and skin of the patient in response to determining that motion data measured by the motion sensor satisfies a motion threshold.

22. The method of claim 15, further comprising by the controller:
receiving, via the transceiver, from the external computing device an indication by the HCP to stop patient data collection; and
in response to receiving the indication, preventing storing in memory at least some of the patient data received from the sensor subsequent to the indication.

23. The method of claim 22, wherein the authorization includes a first time stamp indicating a start of a patient data collection episode, wherein the indication includes a second time stamp indicating an end of the patient data collection episode, and wherein the method comprises associating patient data stored after receiving the first time stamp and before receiving the second time stamp as being associated with the patient data collection episode.

24. (canceled)
